(19) United States
(12) Patent Application Publication
(10) Pub. No.: US 2010/0093050 A1
(43) Pub. Date: Apr. 15, 2010

(54) BIOTECHNICAL AND MICROBIOLOGICAL PRODUCTION METHOD AND EQUIPMENT

(76) Inventor: Elias Eino Hakalehto, Kuopio (FI)

Correspondence Address: PILLSBURY WINTHROP SHAW PITTMAN, LLP, P.O. BOX 10500, MCLEAN, VA, 22102, US

(21) Appl. No.: 12/522,258

(22) PCT Filed: Jan. 4, 2008
(86) PCT No.: PCT/FI08/00001

§ 371 Date: Oct. 8, 2009

(30) Foreign Application Priority Data
Jan. 4, 2007 (FI) 20070008

(51) Int. Cl. C12P 1/04; C12M 1/00; C12M 1/36

(52) U.S. Cl. 435/170; 435/289.1; 435/286.1

(57) ABSTRACT

A method and an apparatus enabling the simultaneous cultivation and product formation in both aerobic and anaerobic conditions of microbes or other production organisms in the same bioreactor. The used microbes may be facultatively anaerobic bacteria, for example.

### BACKGROUND FOR THE INVENTION

In utilizing and studying microbes in industry or medicine or in environmental cleaning, the used microbiological and bacteriological methods in most cases call for the culturing of cells or equivalent in a nutrient substrate before being able to show them, clarifying the effects of their action or getting to a desired production result. This production result may be a cell growth, e.g. for feed or protein, or the formation of one or several desired metabolic products. These products can be in liquid or gas form or they can be separated from a production liquid into solid form by precipitation. The production result of a microbiological reaction can also be the cleaning of environment, like soil or water, or the elimination of a harmful substance from an organism.

Nutrient substrates contain vital nutrients for microbes and they are designed to be as suitable as possible for the microbes and other organisms to be studied. Plant and animal cells have been started to generally be cultivated with similar methods as microbes. Conventionally, nutrient substrates are divided into general nutrient substrates and selective nutrient substrates. In the former many different microbes can widely be cultivated whereas selective nutrient substrates choose or enrich selective species or strains. Studied microbes may be for example single cell bacteria or yeasts, or filamentous molds, or algae or protozoa.

Microbe cultivations and studies may be related to projects for example in medicine, health care, pharmaceutical industry, food, chemistry or cosmetics industries or forestry industry. They may also be part of the control of building mold damage or the follow-up of environmental condition to find out the quality of water, air or other environmental quality. Normally these cultivations take place in thermostatically controlled cabinets (thermal cabinets or incubators) or rooms, which thermostated cabinets are located in laboratories or equivalent. Often there is need to collect microbe samples or equivalent in the field or as part of field experiments or on site for example in production processes, different locations in hospitals or canteens. Large quantities of microbe cells are generally tried to be cultivated in different kinds of bioreactors, fermentors. Microbiological production reactions often also take place in these. In studies performed by Finnoflag Oy (Kuopio and Siilinjärvi, Finland), it has been shown that microbes can efficiently be cultivated in controlled environments in a cultivation case in a way that air or gas is led into the sampling and cultivation syringes in the case (Finnish patent no FI 106561).

In the biotechnical industry fermentation has conventionally been used for cultivating microbes or carrying out a production method by them. Although “fermentation” has originally meant anaerobic fermentation, this concept nowadays includes all microbe action that is carried out in bioreactors. This action may happen just as well in the presence of oxygen, aerobically, or without oxygen, anaerobically. In this application the term “reactor” means bioreactor.

In fermentation a device in which the biotechnical or microbiological reaction happens is called a bioreactor or fermentor. Originally it was used for microbe reactions like the reactions needed in the production of microbe cell mass, metabolism products, enzymes, antibiotics or other products carried out by bacteria, yeasts or molds. Later also euchariotic organisms, plant or animal cells have been used in these reactions. A bioreaction or fermentation can be carried out except for forming a product, also to clean a material, for example by breaking up toxins or by consuming nutrients. Thus all kinds of biological refineries are also bioreactors. The form of a bioreactor may be a barrel-like container or pipe or tube or a tray-like surface or a bottle or can shaped container or a basin or tank or equivalent. Essential is that a product or products are formed using a biocatalyst or a desired service, like cleaning water in a biological refinery or the binding of nitrogen to soil using nitrogen-binding bacteria, is carried out. The form of a bioreactor may partly be round as in Finnish patent no FI 115967 or like an injection syringe as in Finnish patent no FI 106561.

One important form of a bioreactor is the cylindrical form. It contains a mixing device, normally some kind of a propeller. In this case the reactor, i.e. fermentor is called also with the term STR (stirred tank reactor). If the cultivation is continuous the reactors name can be CSTR (continuous stirred tank reactor). In this case fresh nutrient substrates are added to the reactor container continuously and correspondingly reaction liquid is removed continuously. When cultivating aerobic microbes the nutrient substrate, i.e. liquid in a reactor is normally aerated by pumping air into it for example by a tube pump. If this mixing of liquid nutrient substrates takes place by using air and not for example mechanically, the reactor is called an “air lift” fermentor. When a biocatalyst, which can be cells, cell tissue, mycelium growth of a microbial mushroom (mold) or bacteria (e.g. hyphal forms), cell parts or structures or enzymes, is not situated freely in the reactor liquid but is attached to some carrier material in immobilized form and air is led to the reactor and reactor liquid, a fluidized bed fermentor is formed.

If a hollow structure, for example a tube is used as a reactor, one can talk of a hollow fiber type of a fermentor. All in all the structure of the reactor may be any solution that is applicable to the bioreaction to be carried out. The volume of the reactor/fermentor may vary according to the task. Normally the smallest reactors are just one liter laboratory fermentors, the biggest can be large tanks with capacity of hundreds of cubic metres. Also water refinery basins are bioreactors as well as basins in which single cell proteins are produced under sun light with the help of algae.

In aerobic fermentation the basis is to prevent gas that contains oxygen of getting into the bioreactor. The mixing is normally done mechanically. As a result of their metabolism, anaerobic bacteria normally form gases and thus excess pressure into the reactor which helps its content stay anaerobic. Because of gases and liquid pressures, the movements of these or the mechanical operation of the device, such as the movement of stirrers, different so-called shear forces are formed in the reactor. These cause strain to the microbes or cells or equivalent in the liquid, suspension, reactor mixture, culture or equivalent in the reactor, which can also be called environmental stress. In many cases it has been established that shear forces promote the attachment of microbes or cells or equivalent onto surfaces as biofilms. By environmental stress one can also mean the sum of all environmental effects which decreases the viability or functionality of microbes or cells or equivalent in a reactor or culture.

When nutrients and reaction products are continuously added and removed from the reactor during the reaction, the bioreactor is called continuously operating and the corresponding single cultivation batch cultivation or batch culture. The continuous operation of the reaction often improves its adjustability and the formation of products, their yield and productivity. Organisms, that possibly can be used as biocatalysts in the reactor, can be aerobic, or microaerophilic, which need only small amounts of oxygen, or facultatively anaerobic, that can grow and metabolise both with the presence of oxygen and without it, or oblicately anaerobic, which do not tolerate oxygen. Facultatively anaerobes are among others enteric bacteria, for example the bacteria from the genera Escherichia, Salmonella, Klebsiella, Citrobacter, Aerobacter, Serratia and Enterobacter. Of these, for example Escherichia species anaerobically form especially lactic acid, acetic acid and succinic acid, ethanol and formic acid (or hydrogen and carbon dioxide) (so called mixed acid fermentation), whereas for example Enterobacter species produce additionally 2,3-butanediol and more ethanol and considerably less acid metabolic products (so called butanediol fermentation). Facultatively anaerobic bacteria also include pathogenic strains such as Salmonella and Vibrio cholerae strains.

In many biotechnical reactions microbe cells and other cells are produced in different conditions compared with the conditions in which the actual formation of the product optimally happens. Correspondingly, the pretreatment of raw materials, such as enzymatic hydrolysis, can require different conditions than the actual production reaction in which the raw material is worked up into a final product such as biofuel, solvent, acid, basic chemical or other equivalent product.

Microbes can be cultured as pure cultures, in which case the entire microbe population represents the same microbe strain, or as mixed cultures. In the latter option one can exploit the influence that different microbes together or in cooperation can achieve. In this case the environmental conditions have to be adjusted so that the desired production or product forming conditions are realized.

### DESCRIPTION OF THE INVENTION

In a bioreactor it can be advantageous to cultivate in one part in aerobic conditions a specific microbe population and in another part anaerobically either the same or a specific other microbe population. With the help of this approach, in the aerobic part of the bioreactor one can reach maximum yield for example in the case of cell growth, and cells or substances suitable for microbe nutrients that have preparatively been broken down by cells, move into the anaerobic part. In the anaerobic parts, metabolic products that can start to hinder the production or the continuous formation of the product through different genetic and biochemical adjustment mechanisms, either move to the aerobic part or are collected from the bioreactor. When using the method according to the present invention the shape of the reactor can be anything related to different applications of use.

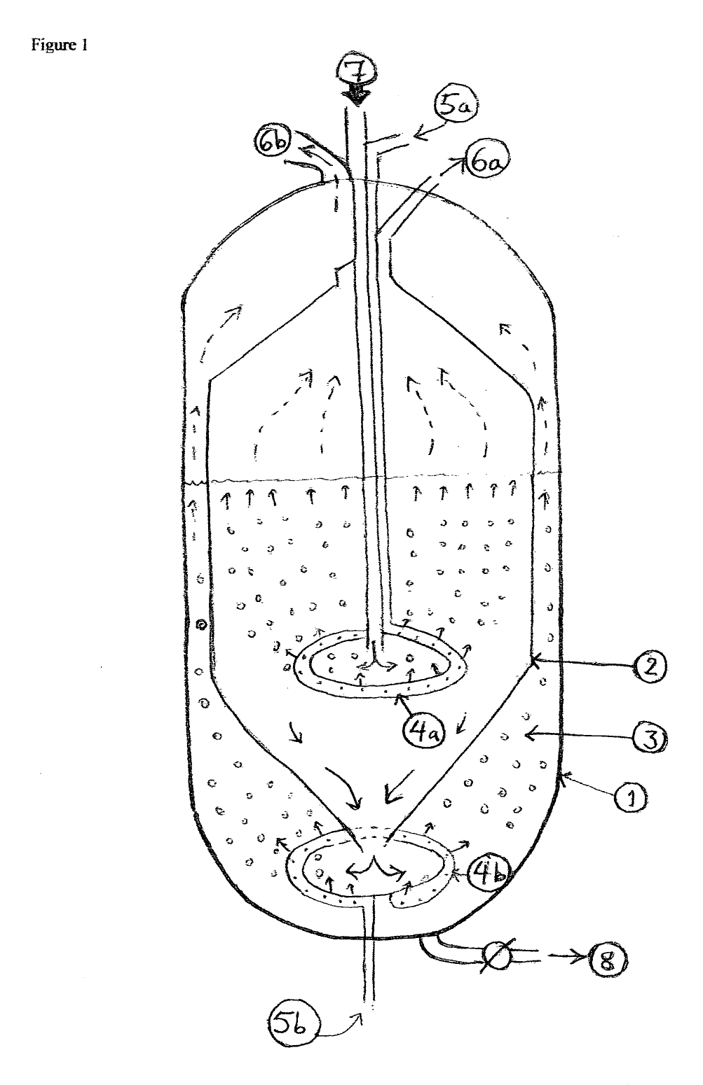

To exploit the observation that the anaerobic growth and metabolism of microbes has, in our test set-ups, been comparable in speed to their aerobic growth and metabolism, we have designed a bioreactor solution, which is described in this patent application (FIG. 1). A special feature in this is that gas is led into different parts of the reactor liquid or reactor mixture in the reactor and that the oxygen content can be 0-100%. Further: in order to gain the best result, different gas or gas mixture can be lead to different parts of the reactor.

As gases that are led into the reactor liquid or reactor mixture bubble they cause liquid and gas phase interfaces. The forming of these, speed up the movement of materials. Thus on the one hand, one gets nutrients quickly for the cells to use and on the other hand waste products are moved away from the cells. Taking these considerations into account and because optimal conditions for cell growth, cell respiration product forming, enzymatic reactions, fermentation reactions and the breaking up of different substances etc. can be effectuated in different parts of the liquid or mixture, offers the method and equipment according to the present invention a possibility of utilizing in a new and more efficient way all of the biocatalyst potential that microbe cells or equivalent have to offer. Additionally essential is that the flow of gases in different parts of the reactor may each time be adjusted directly according to the available measurement results in order to gain the best outcome of the process.

Thus for example the cultivation of Klebsiella sp. strain that forms butanediol can be carried out in the reactor's aerobic part after which the cells that drift to the microaerobic or anaerobic parts and can carry out the formation of the actual product, 2,3-butanediol. With this approach it is possible in some cases to avoid the need for immobilizing the biocatalyst, for example, which in part produces cost savings. When returning back to the aerobic area, the Klebsiella cells of this example can continue their multiplication, and thus increase the biocatalyst formation.

In order to optimize the usability of different reactor solutions, different parts of the reactor have to be screened by measurements. These measurements may include e.g. pH, pO₂, pCO₂, optical density, analyte concentrates etc. It is of particular importance that these pieces of information allow correct adjustments of the gas flows to different parts of the reactor.

The method and apparatus according to the present invention are particularly specifically suitable for exploiting the facultatively anaerobic bacteria. For example, by their help it is possible to produce organic acids, alcohols or 2,3-butanediol for the needs of chemical, food or polymer industries. These organisms are able to ferment anaerobically or microaerobically by using mixed acid fermentation or butanediol fermentation route. Bacteria of the genera Klebsiella or Enterobacter, for instance, having the capability to fix atmospheric nitrogen, can utilize the latter metabolic pathway. When fixing atmospheric nitrogen in the aerobic part of the reactor, it can be exploited in anaerobic fermentation as the bacteria that act as biocatalysts move to the anaerobic part of the reactor. In this patent application the words reactor, bioreactor and fermentor have the same meaning.

The method according to the present invention could be exploited also when different microbes are cultivated as mixed cultures. By creating different gas exchange conditions into different parts of the fermentor it is possible to achieve optimal conditions for the product formation into areas where enhanced microbe function is required.

To exploit the present invention, one advantageous bioreactor model is presented in FIG. 1. When using it, two or more gases are directed via two or more routes into the reactor chamber (1) or basin or equivalent, in such a way that part of the gases are aerobic (contain oxygen) (4a, 4b) and another part are anaerobic (without oxygen) (4b, 5b). This is especially suitable for cultivating and exploiting facultatively anaerobic bacteria but also for the cultivation and utilization of many other microbes, such as some moulds.

The gases are led either entirely or in part into the solution, suspension, reaction mixture, culture or equivalent (3). The bioreactor may contain different compartments and have impermeable or partially permeable middle walls in between them. The exhaust gases coming out from the bioreactor or the cultivation chamber, and with them liberated liquids for example in aerosol form, or solid substances, that may be for example as particles (6a, 6b), are collected as usable products. The products are collected from the solution, suspension, reaction mixture, culture or equivalent (8), which is removed from the reactor. The conditions inside the reactor can be adjusted by the controlling unit according to the measurement data from the outgoing substance concentrations or characteristics obtained by measurements based on sensor data. The nutrients and additives can be fed advantageously into the aerobic reactor first through the inlet opening (7), from where they, for example by gravitation, settle down into the anaerobic part of the reactor. More than one inlet for nutrients or raw materials may also be used.

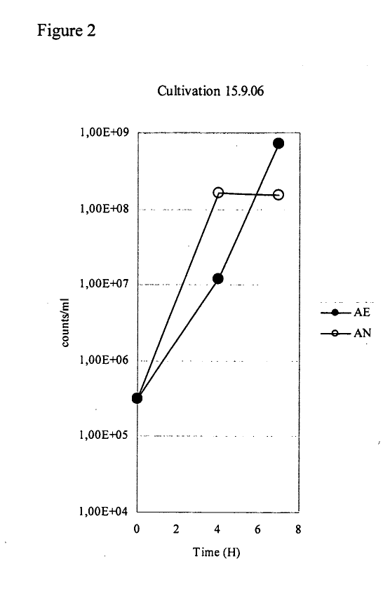

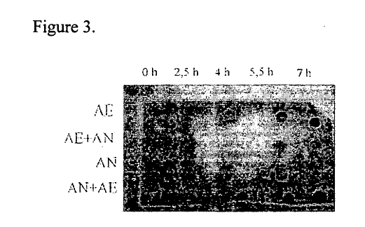

Example 1. One example, in this case of the different activities of one microbe Salmonella enterica serovar Enteritidis in aerobic and anaerobic conditions, can be seen in the growth curve (FIG. 2) and dot blot immunoassay picture (FIG. 3). The bacterium in question (strain IHS 59813) was cultivated in a PMEU equipment (Portable Microbe Enrichment Unit, Finnoflag Oy, Kuopio and Siilinjärvi, Finland). Before inoculating the cultivation syringes of the enrichment unit, the bacterial inoculant strain was made younger into a culture in TYG (Tryptone-Yeast extract-Glucose) broth 24 hours before the onset of the experiment. Salmonella culture was taken into the TYG medium in the cultivation syringes as a final concentration of 10.000-100.000 CFU/ml by visual estimation.

The cultivations were carried out as follows:

Syringe 1: Aerobic culture in the PMEU enrichment equipment, air flow 100%, temperature +37° C. for 2 hours, after that +40° C. until the end of the experiment.
Syring 2: Aerobic culture in the PMEU enrichment equipment, air flow 100%, temperature +37° C. for 2 hours, after that +40° C., after 3 hours from the onset of the cultivation the syringe switched into another PMEU equipment for anaerobic cultivation.
Syringe 3: Anaerobic culture using nitrogen gas in the PMEU enrichment equipment, gas flow adjusted visually to the same as in the aerobic case, temperature +37° C. for 2 hours, after that +40° C. until the end of the experiment.
Syring 4: Anaerobic culture using nitrogen gas in the PMEU enrichment equipment, gas flow adjusted visually to the same as in the aerobic case, temperature +37° C. for 2 hours, after that +40° C., after 3 hours from the onset of the cultivation the syringe switched into another PMEU equipment for aerobic cultivation

Samples Collected during Cultivation
0 h to plate culture from the bacterial suspension applied to the syringes, and from each syringe to dot blot analysis
2.5 h to dot blot analysis
4 h to plate culture and dot blot analysis
5.5 h to dot blot analysis
7 h to plate culture and dot blot analysis

Plate Cultures:

Samples were diluted to sterile water and inoculated to TYG plates. The plates were incubated in an incubator chamber for 24 hours at +40° C. and then the colonies were counted. The growth curves were drawn accordingly (FIG. 2). In this experiment, against general belief that anaerobic metabolism would be slower than aerobic, with the PMEU equipment, a faster initial bacterial growth was achieved anaerobically than by aerobic metabolism (0-4 h). After that the growth attenuated probably due to the exhaustion of the available nutritive substances. Therefore, already after 8 hours the production of bacterial biomass is on a higher level in the aerobic PMEU cultivation syringe than in the anaerobic one.

Dot Blot Analysis

The samples from different time points were applied to nitrocellulose filter paper strip. The strip was allowed to dry up, after which it was preserved in a refrigerator (7 days) until the analysis.

The strip was moved for blocking into the BSA-TBS—solution, where it was kept in a shaker for 1 hour. After that the strip was incubated in an antibody solution (H463 12.3.98, dilution 1:70 in 1×TBS) for 1 hour. After the antibody bath the strip was washed 3×10 min in a washing solution (1.5% milk powder/ 0.5 Tween in TBS).

Then the strip was incubated in the secondary antibody solution (AP-Goat Anti-Rabbit IgG, dilution 1:1000 in TBS) for 1 hour. The washes were repeated 2×10 min in the washing solution and 1×10 min in the Afos buffer solution. In the end the strip was stained in the staining solution (NTB+BCIP in Afos buffer solution). The stained strip is seen in FIG. 3. Aerobically grown bacterial samples from different time points (AE) and the anaerobic samples from different time points (AN) indicate different fashions for the formation of the fimbrial attachment threads in these different conditions. In the anaerobic cultivation the maximal fimbrial synthesis took place at 5.5 h with their expression attenuating after that, whereas in the aerobic process the production of the protein in question remained on a high level also after that. When the culture was started aerobically and switched to the anaerobic mode (AE+AN), or vice versa (AN+AE), the production was delayed when the conditions changed.

When the plate results from the PMEU cultivations were studied, it was found out that the anaerobic cultivation had produced faster cell number increase between 0-4 hours, which was a surprising result, because aerobic metabolism has generally been considered faster than anaerobic (FIG. 2). This common belief is based on the cellular production of two ATP moles per one mole of glucose in for instance homolactic fermentation, whereas in aerobic metabolism they generate 28 moles. On the basis of the research behind the present invention it seems to be so, that in the anaerobic fermentation the availability of the nutrients and the removal of diffusion limitations could replace the shortness of the metabolic route.

Example 2. In a similar way as in the example 1 Escherichia coli (ATCC 25922) and Klebsiella mobilis (ATCC 13048) bacterial strains were studied anaerobically, microaerobically and aerobically with the PMEU equipment. It was found out that the latter strain formed 2,3-butanediol already after some hours of cultivation, and in such a way that the butanediol formation continued also after the bacterial growth had become slower. In microaerobic cultures a gas mixture of 5% O₂, 10% CO₂, and 85% N₂ was used. The growth produced in the aerobic and microaerobic pure cultures is seen in Table 1. In Table 2 the aerobic growth produced by the Escherichia coli (ATCC 25922) and Klebsiella mobilis (ATCC 13048) mixed cultures is presented. An interesting observation was that when in both experiments TYG (Tryptone-Yeast extract-Glucose) broth was used as growth medium, both the pure and mixed cultures gave equal yields for both bacterial strains. This indicated that these bacterial strains did not compete on the nutrients in the given circumstances. Nitrogen gas was used in the anaerobic cultivation. The plate cultivations were carried out as described in Example 1, the growth medium being Chromagar TM Orientation Medium (Becton Dickinson, USA), pH measurements were carried out with Orion Model 420A pH meter (Thermo Electron Corporation, USA).

TABLE 1
Microbe cultivations aerobically at +37° C. (1-5)
and microaerobically in the same temperature (1M-5M).

| Samples | dilution −4 | dilution −5 | dilution −6 | dilution −7 | dilution −8 |
|---|---|---|---|---|---|
| 0 hours | | | | | |
| 1 and 1 | 104 | 28 | | | |
| M | 257 | 36 | | | |
| 2 and 2 | 350 | 40 | | | |
| M | 365 | 59 | | | |
| 3 and 3 | 82 | 14 | | | |
| M | — | — | | | |
| 7 hours | | | | | |
| 1 | | | | 20 | 6 |
| | | | | — | 2 |
| 2 | | | | 120 | 21 |
| | | | | 130 | 23 |
| 3 | | | | 130 | 16 |
| | | | | — | 14 |
| 1 M | | | | 54 | 6 |
| | | | | 68 | 9 |
| 2 M | | | | 67 | 10 |
| | | | | 77 | 4 |
| 3 M | | | | 88 | 4 |
| | | | | 81 | 3 |

The used strains:
1. Escherichia coli
2. Klebsiella mobilis ATCC 13048
3. Klebsiella oxytoga III a8 E81
4. Klebsiella pneumoniae ssp pneumoniae IIIa8 E102
5. Klebsiella pneumoniae ssp pneumoniae IIIa2 E111.

TABLE 2
Mixed cultivation of Escherichia coli (E.c) and
K. mobilis (K.m.) sp. strains at 37° C. in the PMEU.

| | | Cells | Cells | pH | Cells | pH |
|---|---|---|---|---|---|---|
| Culture | Strain | after 2.5 h | after 4 h | after 4 h | after 6 h | after 6 h |
| 1 | E.c. | 1.8 × 10exp7 | 1.9 × 10exp8 | 5.6 | 1.7 × 10exp9 | 6.6 |
| | K.m. | 1.0 × 10exp7 | 2.3 × 10exp8 | 5.6 | 2.4 × 10exp9 | 6.6 |
| 2 | E.c. | 5.1 × 10exp7 | 1.1 × 10exp7 | 5.5 | 1.3 × 10exp9 | 5.9 |
| | K.m. | 6.0 × 10exp6 | 1.0 × 10exp7 | 5.5 | 7.0 × 10exp8 | 5.9 |
| 3 | E.c. | 4.5 × 10exp7 | 1.2 × 10exp8 | 5.5 | 1.1 × 10exp9 | 6.3 |
| | K.m. | 5.1 × 10exp7 | 1.4 × 10exp8 | 5.5 | 1.0 × 10exp9 | 6.3 |
| 4 | E.c. | 3.7 × 10exp7 | 7.7 × 10exp7 | 5.6 | 4.0 × 10exp8 | 5.9 |
| | K.m. | 6.5 × 10exp6 | 4.0 × 10exp7 | 5.6 | 1.0 × 10exp8 | 5.9 |
| 5 | E.c. | 1.0 × 10exp7 | 4.1 × 10exp7 | 5.4 | 9.0 × 10exp8 | 5.8 |
| | K.m. | 1.5 × 10exp7 | 8.9 × 10exp7 | 5.4 | 1.7 × 10exp9 | 5.8 |
| 6 | E.c. | 1.1 × 10exp7 | 1.2 × 10exp8 | 5.5 | 6.0 × 10exp8 | 5.5 |
| | K.m. | 3.0 × 10exp6 | 4.5 × 10exp7 | 5.5 | 7.5 × 10exp8 | 5.5 |

The inoculated cell concentrations were about 5 × 10exp6 for E. coli and for K. mobilis about 1 × 10exp6 (in cultures 1, 3 and 5) or about 5 × 10exp5 (cultures 2, 4 and 6) per ml of the medium. The initial pH of the TYG liquid medium was 6.8 (samples 1-4) and 6.14 (samples 5 and 6).

1. A method for carrying out bioreaction characterized in that in the reaction, anaerobic or microaerobic bacterial metabolism and growth occur equally fast as the aerobic growth and metabolism in the same bacteria.

2. A method according to the claim 1, characterized in that different gases are led into a reactor in a controlled fashion so that the yield and productivity of an anaerobic or microaerobic fermentation is improved with the aid of the gas.

3. A method according to the claim 2, characterized in that an oxygen-free gas mixture is led into a solution, suspension, reaction mixture, culture or equivalent in the reactor, and the gas mixture contains an inert gas for flushing out or for collecting the evaporating microbial or other cellular reaction products or waste substances or other evaporating substances in such a way that the anaerobic or microaerobic fermentation yield or productivity is increased with the inert gas.

4. A method according to the claim 3, characterized in that air or an oxygen-containing gas is led into the reactor in such a way that the reactor has both an aerobic part and an anaerobic part.

5. A method according to claim 2, characterized in that all gases led to the reactor or a part of the gases are sterilized.

6. A method according to claim 3, characterized in that the gases led to the reactor form bubbles into the solution, suspension, reaction mixture, culture or equivalent.

7. A method according to the claim 6, characterized in that the solution, suspension, reaction mixture, culture or equivalent in the reactor partially contains oxygen and is partially oxygen-free, or that in different parts of the reactor, different partial pressures of gases are maintained on purpose.

8. A method according to claim 3, characterized in that substances liberated in a gaseous form from the solution, suspension, reaction mixture, culture or equivalent are led out as a uniform gas mixture.

9. A method according to claim 3, characterized in that substances liberated in a gaseous form from the solution, suspension, reaction mixture, culture or equivalent are led out as two or more gas mixtures with different compositions using two or more different routes.

10. A method according to the claim 9, characterized in that gaseous, liquid or solid substances are collected for exploitation from the one or more gas flows out of the reactor.

11. A method according to claim 2, characterized in that physical conditions of the gases led to the reactor from two or more sources are different when compared to each other.

12. A method according to claim 3, characterized in that substances can be added into or taken out from the solution, suspension, reaction mixture, culture or equivalent continuously.

13. A method according to claim 1, characterized in that the bacteria are used as biocatalysts in the bioreaction.

14. A method according to claim 1, characterized in that facultatively anaerobic bacteria are used as production organisms in the bioreaction or fermentation in a bioreactor.

15. A method according to the claim 14, characterized in that enteric bacteria or enterobacteria are used as the production organisms.

16. A method according to the claim 15, characterized in that the production organisms belong to the genera Klebsiella or Enterobacter or any other genera using butanediol fermentation in their metabolism.

17. A method according to claim 14, characterized in that the bacterium used as production organism is capable of fixing the atmospheric nitrogen in an aerobic part of the bioreactor, and atmospheric nitrogen is exploited also in an anaerobic fermentation.

18. An apparatus for carrying out the method according to claim 1, characterized in that the apparatus consists of a reactor having a reaction chamber or basin or equivalent, into which two or more gases are led via two or more routes in such a way that a part of the gases are oxygen-containing and a part are oxygen-free.

19. An apparatus according to the claim 18, characterized in that the gases are led entirely or partially into a solution, suspension, reaction mixture, culture or equivalent in the reactor.

20. An apparatus according to the claim 18, characterized in that it contains different compartments and impermeable or partially permeable middle walls for substances in between them.

21. An apparatus according to claim 18, characterized in that outgoing gases from the reaction chamber, and liberated liquids, or solid substances are collected as usable products.

22. An apparatus according to claim 18, characterized in that products are collected from an outgoing solution, suspension, reaction mixture, culture or equivalent removed from the reactor.

23. An apparatus according to claim 18, characterized in that conditions inside the reactor are adjusted by a control unit according to sensor measurement data obtained from the outgoing substances from the reactor.
